(19) United States
(12) Patent Application Publication
(10) Pub. No.: US 2024/0335534 A1
(43) Pub. Date: Oct. 10, 2024

(54) METHODS OF REDUCING PARTICLE FORMATION AND COMPOSITIONS FORMED THEREBY

(71) Applicant: Regeneron Pharmaceuticals, Inc., Tarrytown, NY (US)

(72) Inventors: Mayank PATEL, New City, NY (US); Stacy WASINGER, Hopewell Junction, NY (US); Danya SPRITZER, Briarcliff Manor, NY (US); Xiaolin TANG, Old Tappan, NJ (US)

(73) Assignee: Regeneron Pharmaceuticals, Inc., Tarrytown, NY (US)

(21) Appl. No.: 18/752,089

(22) Filed: Jun. 24, 2024

Related U.S. Application Data
(63) Continuation of application No. 16/100,369, filed on Aug. 10, 2018, now Pat. No. 12,042,538.
(60) Provisional application No. 62/560,365, filed on Sep. 19, 2017.

(51) Int. Cl. A61K 39/395; A61K 9/00; A61K 38/46; A61K 47/26; C07K 16/00

(52) U.S. Cl. CPC A61K 39/39591; A61K 38/465; A61K 47/26; C07K 16/00; A61K 9/0019

(57) ABSTRACT

Biopharmaceutical compositions and drug products disclosed herein exhibit reduced amounts of subvisible particle formation. Compositions and drug products disclosed herein comprise a protein and a surfactant or stabilizer including high percentage amounts of a long-chain fatty acid ester. Also disclosed herein are methods of preparing and storing such compositions and drug products.

### CROSS-REFERENCE TO RELATED APPLICATIONS

This application is a continuation of, and claims the benefit of priority from, U.S. application Ser. No. 16/100,369, filed Aug. 10, 2018, which claims the benefit of priority from U.S. Provisional Application No. 62/560,365, filed on Sep. 19, 2017, each of which is incorporated by reference in its entirety.

### SEQUENCE LISTING

This application contains a sequence listing, submitted electronically in XML format under the filename “00166-0011_Sequence Listing”, which is incorporated by reference herein in its entirety. The XML copy of the sequence listing was created on Jun. 20, 2024, and is 27,931 bytes in size.

### FIELD OF THE DISCLOSURE

This disclosure is directed to biopharmaceutical formulations and drug products exhibiting reduced amounts of subvisible particle formation upon storage, and to methods of their preparation and storage. Specifically, this disclosure is directed to formulations and drug products comprising a protein and a surfactant or stabilizer including high percentage amounts of a long-chain, mono-unsaturated fatty acid ester, and methods of their preparation and storage.

### BACKGROUND

Polysorbates have conventionally been used in drug products (also referred to as “DP”) containing a protein as an active ingredient, to protect proteins from surface-induced (air/liquid or solid/liquid) instability during manufacturing, storage, handling, and administration. It has been discovered that lipases copurified with proteins of interest (POIs), when present in formulations with polysorbates, may hydrolyze the fatty acid esters in polysorbates into free fatty acids. Non-enzymatic hydrolysis of fatty acid esters in polysorbates may also occur in formulations at a slower rate. The free fatty acids formed as a result of hydrolysis may aggregate and form particulates in drug products containing such formulations over time. Particulates (both visible and subvisible) can impact product stability, reduce a drug product's shelf life because of its failure to meet compendial particulate matter specifications (e.g., U.S. FDA specifications), and may have clinical effects, such as an immunogenic reaction upon administration.

Hydrophobic interaction chromatography (HIC) or affinity chromatography of POIs may reduce or remove lipases that are co-purified with the POIs, thus decreasing hydrolysis of fatty acid esters. However, addition of a HIC or affinity chromatography step to the preparation of protein formulations requires adding, for example, equipment, materials, preparation, protocol, and protocol validation to manufacturing methods, which results in added time and costs. In addition, a HIC or affinity chromatography step to remove enzymes will not aid in preventing non-enzymatic hydrolysis of fatty acid esters in formulations. A method of reducing or preventing subvisible and visible particulate formation in protein compositions, for example, without the use of added HIC or affinity chromatography steps, is therefore desired.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments. Any features of an embodiment or example described herein (e.g., composition, formulation, method, etc.) may be combined with any other embodiment or example, and are encompassed by the present disclosure.

FIG. 1 is a diagram of the chemical structure of polyoxyethylene (20) sorbitan monooleate, the predominant fatty acid ester in polysorbate 80.

FIG. 2 is a chart depicting the number of subvisible particulates (≥10 μm) measured by the membrane microscopic method, in protein drug products comprising various types of polysorbate 80.

FIG. 3 is a chart depicting the number of subvisible particulates (≥10 μm) measured by micro-flow imaging (MFI), in protein drug products comprising various types of polysorbate 80.

FIG. 4 is a chart depicting the measured concentration of free fatty acids in protein drug products comprising various types of polysorbate 80.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” For the terms “for example” and “such as,” and grammatical equivalences thereof, the phrase “and without limitation” is understood to follow unless explicitly stated otherwise.

As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

It should be noted that all numeric values disclosed herein (including all disclosed values, limits, and ranges) may have a variation of +/−10% (unless a different variation is specified) from the disclosed numeric value. Moreover, in the claims, values, limits, and/or ranges means the value, limit, and/or range +/−10%.

### DETAILED DESCRIPTION

This disclosure is not limited to the particular compositions, formulations, material manufacturer, drug products, methods, or experimental conditions disclosed herein, as many variations are possible within the purview of one of ordinary skill in the art. The terminology used herein is for the purpose of describing particular embodiments only, and is not intended to be limiting.

Unless defined otherwise, all technical and scientific terms used herein have the same meaning as is commonly understood by one of ordinary skill in the art to which this disclosure belongs. Although any methods and materials similar or equivalent to those described herein can be used in the practice or testing of the present invention, particular methods and materials are now described. All publications mentioned are hereby incorporated by reference.

The term “protein” as used herein refers to any amino acid polymer having more than about 20 amino acids covalently linked via amide bonds. Proteins contain one or more amino acid polymer chains, generally known in the art as “polypeptides.” Thus, a polypeptide may be a protein, and a protein may contain multiple polypeptides to form a single functioning biomolecule of a single conformation. Disulfide bridges (e.g., between cysteine residues to form cystine) may be present in some proteins. For example, disulfide bridges are essential to proper structure and function of insulin, immunoglobulins, protamine, and the like.

In addition to disulfide bond formation, proteins may be subject to other post-translational modifications. Those modifications include lipidation (e.g., myristoylation, palmitoylation, farnesoylation, geranylgeranylation, and glycosylphosphatidylinositol (GPI) anchor formation), alkylation (e.g., methylation), acylation, amidation, glycosylation (e.g., addition of glycosyl groups at arginine, asparagine, cysteine, hydroxylysine, serine, threonine, tyrosine, and/or tryptophan), and phosphorylation (i.e., the addition of a phosphate group to serine, threonine, tyrosine, and/or histidine).

As used herein, the term “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, antibody-like molecules, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells).

The term “antibody,” as used herein, includes immunoglobulins comprised of four polypeptide chains, two heavy (H) chains and two light (L) chains inter-connected by disulfide bonds. Typically, antibodies according to the present disclosure have a molecular weight of over 100 kDa, such as between 130 kDa and 200 kDa, such as about 140 kDa, 145 kDa, 150 kDa, 155 kDa, or 160 kDa. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3.

A class of immunoglobulins called Immunoglobulin G (IgG), for example, is common in human serum and comprises four polypeptide chains—two light chains and two heavy chains. Each light chain is linked to one heavy chain via a cystine disulfide bond, and the two heavy chains are bound to each other via two cystine disulfide bonds. Other classes of human immunoglobulins include IgA, IgM, IgD, and IgE. In the case of IgG, four subclasses exist: IgG 1, IgG 2, IgG 3, and IgG 4. Each subclass differs in their constant regions, and as a result, may have different effector functions.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcTR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcTR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein may be an Fc-containing protein and may exhibit enhanced serum half-life (as compared with the same Fc-containing protein without the recited modification(s)), and may have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 259I (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); or a 307 and/or 308 modification (e.g., 308F or 308P).

The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusiani, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g. a retinal cell that expresses a viral gene (e.g., a PER.C6™ cell).

The term “fatty acid ester” means any organic compound that contains a fatty acid chain linked to a head group via an ester bond. An ester bond is formed when a hydroxyl group (e.g., an alcohol or carboxylic acid) is replaced by an alkoxy group. As used herein, the hydroxyl group can be part of a carboxylic acid, more specifically a fatty acid, and/or an alcohol, such as glycerol, sorbitol, sorbitan, isosorbide, or the like. The alcohol group is generally referred to herein as the head group.

Examples of fatty acid esters generally include phospholipids, lipids (e.g., the head group is glycerol, including monoglycerides, diglycerides, and triglycerides), and surfactants and emulsifiers, including for example polysorbates like polysorbate 20, polysorbate 60, and polysorbate 80, which are non-ionic detergents. Surfactants and emulsifiers are useful as cosolvents and stabilizers. They function by associating with both a hydrophilic surface and a lipophilic surface to maintain dispersion and structural stability of ingredients, like proteins. Surfactants are added to protein formulations primarily to enhance protein stability against mechanical stress, such as air/liquid interface-induced and solid/liquid interface-induced partial unfolding and self-association. Without a surfactant, proteins may in some cases become structurally unstable in solution, and form multimeric aggregates that eventually become subvisible particles.

The term “fatty acid” or “fatty acid chain” means a carboxylic acid having an aliphatic tail. An aliphatic tail is simply a hydrocarbon chain comprising carbon and hydrogen, and in some cases, oxygen, sulfur, nitrogen and/or chlorine substitutions. Aliphatic tails can be saturated (as in saturated fatty acids), which means that all carbon-carbon bonds are single bonds (i.e., alkanes). Aliphatic tails can be unsaturated (as in unsaturated fatty acids), wherein one or more carbon-carbon bonds are double bonds (alkenes), or triple bonds (alkynes).

Fatty acids are generally designated as short-chain fatty acids, which have fewer than six carbons in their aliphatic tails, medium-chain fatty acids having six to twelve carbons, long-chain fatty acids having thirteen to twenty one carbons, and very long chain fatty acids having aliphatic tails of twenty two carbons and longer. As mentioned above, fatty acids are also categorized according to their degree of saturation, which correlates to stiffness and melting point. Common fatty acids include caprylic acid (8 carbons: 0 double bonds; 8:0), capric acid (10:0), lauric acid (12:0), myristic acid (14:0), myristoleic acid (14:1), palmitic acid (16:0), palmitoleic acid (16:1), sapienic acid (16:1), stearic acid (18:0), oleic acid (18:1), elaidic acid (18:1), vaccenic acid (18:1), linoleic acid (18:2), linelaedic acid (18:2), alpha-linolenic acid (18:3), arachidic acid (20:0), arachidonic acid (20:4), eicosapentaenoic acid (20:5), behenic acid (22:0), erucic acid (22:1), docosahexaenoic acid (22:6), lignoceric acid (24:0), and cerotic acid (26:0).

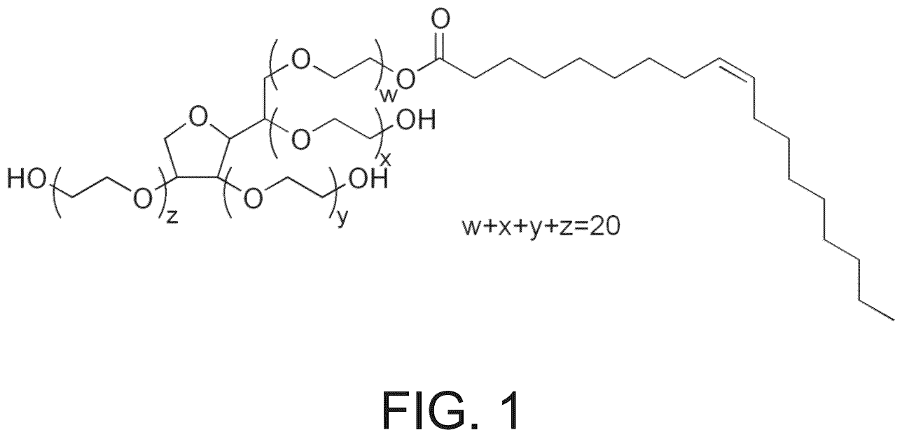

As mentioned above, polysorbates are fatty acid esters useful as non-ionic surfactants and protein stabilizers. Polysorbate 20, polysorbate 40, polysorbate 60, and polysorbate 80 are widely employed in the pharmaceutical, cosmetic, and food industries as stabilizers and emulsifiers. Polysorbate 20 mostly comprises the monolaurate ester of polyoxyethylene (20) sorbitan. Polysorbate 40 mostly comprises the monopalmitate ester of polyoxyethylene (20) sorbitan. Polysorbate 60 mostly comprises the monostearate ester of polyoxyethylene (20) sorbitan. Polysorbate 80 mostly comprises the monooleate ester of polyoxyethylene (20) sorbitan (depicted in FIG. 1).

The quality of commercial grades of polysorbates varies from vendor to vendor. Polysorbates therefore are often mixtures of various chemical entities, consisting mostly of polyoxyethylene (20) sorbitan monoesters (as described above) with, in some cases, isosorbide ester contaminants. They may also include, for example, polyethylene glycol (PEG), intermediate structures, and fatty acid reactants. The head group (in this case polyoxyethylene (20) sorbitan) comprises a sorbitan (a mixture of dehydrated sorbitols, including 1,4-anhydrosorbitol, 1,5-anhydrosorbitol, and 1,4,3,6-dianhydrosorbitol) substituted at three of its alcohol groups to form ether bonds with three polyoxyethylene groups. The fourth alcohol group is substituted with a fatty acid to form a fatty acid ester.

In some commercially available batches of polysorbates, the polysorbate contains isosorbide monoesters. Isosorbide is a heterocyclic derivative of glucose, also prepared by the dehydration of sorbitol. It is a diol, i.e., having two alcohol groups that can take part in the formation of one or two ester bonds. Thus, for example, some lots of polysorbate 80 can contain significant amounts of isosorbide oleate mono- and di-esters.

In addition to head group variation, preparations of polysorbates contain variable amounts of other fatty acid esters. For example, an analysis of one particular source of polysorbate 80 revealed <5% myristic acid, <16% palmitic acid, >58% oleic acid, <6% stearic acid, and <18% linoleic acid. An analysis of another source of polysorbate 80 revealed about 70% oleic acid, with the remainder being other fatty acid esters and impurities. An analysis of yet another source of polysorbate 80 revealed about 86-87% oleic acid. An analysis of a further, more recently-developed source of polysorbate 80 revealed ≥99% oleic acid.

Non-ionic detergents like polysorbate 20 or polysorbate 80 help stabilize large molecules like antibodies and other proteins, and help prevent the formation of oligomeric complexes or other aggregates. Aggregates can become nanoparticles or subvisible particles in the 10 to 100 micron range or 2 to 100 micron range, and interfere with drug product stability and shelf-life, and may induce immunogenicity. Therefore, the stability of protein formulations depends in some cases upon the stability of the non-ionic detergent additive. However, and as is further discussed herein, polysorbate 20 and polysorbate 80 can, in some instances, contribute to the formation of aggregates, nanoparticles, and subvisible particles.

The phrases “subvisible particle” (SVP) and “subvisible particulate” according to the present disclosure refers to a particle that is not visible to the naked eye, especially in a liquid. In other words, a solution or other liquid containing SVPs, but not visible particles, will not appear cloudy. SVPs generally include those particles 100 microns or less in diameter, but in some cases include particles under 150 microns (Narhi et al., “A critical review of analytical methods for subvisible and visible particles,” Curr Pharm Biotechnol 10(4):373-381 (2009)). SVPs may be the result of foreign contaminants, protein aggregation, or aggregation of other components of DP. SVPs may comprise, inter alia, silicone oil droplets (oily droplets), free fatty acids (amorphous particles and/or oily droplets), aggregated protein (amorphous particles), and/or protein/fatty acid complexes (amorphous particles).

SVPs can be detected by any one or more of various methods. The USP standards specify light obscuration and optical microscopy protocols. Other methods include micro-flow image (MFI) analysis, Coulter counting, and submicron particle tracking methods. Several methods of measurement and characterization of SVPs (e.g., light obscuration, flow microscopy, the electrical sensing zone method, and flow cytometry, are discussed in, for example, Narhi et al., “Subvisible (2-100 m) Particle Analysis During Biotherapeutic Drug Product Development: Part 1, Considerations and Strategy,” J. Pharma. Sci. 104:1899-1908 (2015).

Light obscuration is criticized for underestimating protein aggregates and other amorphous structures. Flow image analysis, such as micro-flow imaging (MFI) (Brightwell Technologies, Ottawa, Ontario), is a more sensitive method of detecting the irregularly shaped, fragile, and transparent proteinaceous SVPs, and of differentiating those types of particles from silicone micro-droplets, air bubbles, and other foreign contaminants (Sharma et al., “Micro-flow imaging: Flow microscopy applied to sub-visible particulate analysis in protein formulations,” AAPS J. 12(3): 455-464 (2010)). In general, because SVP measurement and characterization by light obscuration analysis is less sensitive than MFI, particle counts detected by MFI will tend to be higher than particle counts detected by light obscuration analysis. Briefly, MFI is flow microscopy in which successive bright field images are taken and analyzed in real time. Image analysis algorithms are applied to the images to discriminate air bubbles, silicone oil droplets, and proteinaceous aggregates. Volumes as low as about 250 microliters to as high as tens of milliliters can be analyzed. Depending on the system used, particles in the range of two to 300 microns, or one to 70 microns can be detected. (Id.)

The FDA and other government regulatory agencies have placed limits on the amount of SVPs allowed in parenteral drug formulations. The major articulated concern is the uncertainty surrounding potential immunogenicity and downstream negative effects in the patient receiving the drug (Singh et al., “An industry perspective on the monitoring of subvisible particles as a quality attribute for protein therapeutics,” J. Pharma. Sci. 99(8):3302-21 (2010)). For small volume parenteral drugs (e.g., 100 mL or below), the pharmacopeia limits SVPs of greater than or equal to 10 microns to no more than 6,000 SVPs per container, and SVPs of greater than or equal to 25 microns to no more than 600 per container, when determined by light obscuration analysis; and SVPs of greater than or equal to 10 microns to no more than 3,000 SVPs per container, and SVPs of greater than or equal to 25 microns to no more than 300 per container, when determined by the membrane microscopic test. (United States Pharmacopeia and National Formulary (USP 40-NF 28), <787> Subvisible Particulate Matter in Therapeutic Protein Injections.) For ophthalmic drugs, the SVP limits are 50 per mL of 10 microns or greater, 5 per mL of 25 microns or greater, and 2 per mL of 50 microns or greater (Id. at <789> Particulate Matter in Ophthalmic Solutions). Regulatory agencies are increasingly contemplating that manufacturers establish specifications for SVPs of 2 microns or greater (see Singh et al., “An industry perspective on the monitoring of subvisible particles as a quality attribute for protein therapeutics,” J. Pharm. Sci. 99(8):3302-21 (2010)).

The term “esterase” means an enzyme that catalyzes the hydrolysis of an ester bond to create an acid and an alcohol. Esterases are a diverse category of enzymes, including acetyl esterases (e.g., acetylcholinesterase), phosphatases, nucleases, thiolesterases, lipases and other carboxyl ester hydrolases (EC 3.1. As its name implies a carboxyl ester hydrolase (a.k.a. carboxylesterase, carboxylic-ester hydrolase, and EC 3.1.1.1) uses water to hydrolyze a carboxylic ester into an alcohol and a carboxylate. A lipase is a carboxyl ester hydrolase that catalyzes the hydrolysis of lipids, including triglycerides, fats and oils into fatty acids and an alcohol head group. For example, triglycerides are hydrolyzed by lipases like pancreatic lipase to form monoacylglycerol and two fatty acid chains.

Phospholipases are lipases that hydrolyze phospholipids into fatty acids and other products. Phospholipases fall into four broad categories: phospholipase A (including phospholipase A1 and phospholipase A2), phospholipase B, and the phosphodiesterases phosphodiesterase C and phosphodiesterase D. In addition to the canonical phospholipases, phospholipase B-like enzymes, which reside at the lysosome lumen, are thought to be involved in lipid catalysis. For example, phospholipase B-like 2 (PLBL2) is postulated to have esterase activity based upon sequence homology and subcellular localization (Jensen et al., “Biochemical characterization and liposomal localization localization of the mannose-6-phosphate protein p76,” Biochem. J. 402: 449-458 (2007)).

An enzymatic activity associated with the destabilization of polysorbates (including polysorbate 20 and polysorbate 80) has been discovered. That activity was found to be associated with an esterase, such as a polypeptide comprising the amino acid sequences of Table 1. A BLAST search of those peptide sequences revealed identity with a putative phospholipase B-like 2 (PLBL2). PLBL2 is highly conserved in hamster, rat, mice, human and bovine. The applicants envision that PLBL2, which copurifies under certain processes with some classes of proteins-of-interest (POIs) manufactured in a mammalian cell line, has esterase activity responsible for the hydrolysis of polysorbate 20 and 80. Applicants envision that other esterase species, of which PLBL2 is an example, may contribute to polysorbate instability, depending upon the particular protein-of-interest and/or genetic/epigenetic background of the host cell.

Ester hydrolysis of polysorbate 80 was recently reported (see Labrenz, S. R., “Ester hydrolysis of polysorbate 80 in mAb drug product: evidence in support of the hypothesized risk after observation of visible particulate in mAb formulations,” J. Pharma. Sci. 103(8):2268-77 (2014)). That paper reported the formation of visible particles in a formulation containing IgG. The author postulated that the colloidal IgG particles formed due to the enzymatic hydrolysis of oleate esters of polysorbate 80. Although no esterase was directly identified, the author speculates that a lipase or tweenase copurified with the IgG, which was responsible for degrading the polysorbate 80. (Id. at 7.) As stated in that paper, the formation of particles due to the presence of polysorbate 80 is a cause of concern, as such particles may affect the stability and efficacy of the IgG drug product.

TABLE 1

| Sequence | |
|---|---|
| Identifier | Amino acid Sequence |
| SEQ ID NO: 1 | DLLVAHNTWNSYQNMLR |
| SEQ ID NO: 2 | LIRYNNFLHDPLSLCEACIPKP |
| SEQ ID NO: 3 | SVLLDAASGQLR |
| SEQ ID NO: 4 | DQSLVEDMNSMVR |
| SEQ ID NO: 5 | QFNSGTYNNQWMIVDYK |
| SEQ ID NO: 6 | QGPQEAYPLIAGNNLVFSSY |
| SEQ ID NO: 7 | SMLHMGQPDLWTFSPISVP |
| SEQ ID NO: 8 | YNNFLHDPLSLCEACIPKPNA |
| SEQ ID NO: 9 | LALDGATWADIFK |
| SEQ ID NO: 10 | LSLGSGSCSAIIK |
| SEQ ID NO: 11 | YVQPQGCVLEWIR |
| SEQ ID NO: 12 | RMSMLAASGPTWDQLPPFQ |
| SEQ ID NO: 13 | SFLEINLEWMQR |
| SEQ ID NO: 14 | VLTILEQIPGMVVVADADKTED |
| SEQ ID NO: 15 | VRSVLLDAASGQLR |
| SEQ ID NO: 16 | LTLLQLKGLEDSYEGR |
| SEQ ID NO: 17 | MSMLAASGPTWDQLPPFQ |
| SEQ ID NO: 18 | VTSFSLAKR |
| SEQ ID NO: 19 | QNLDPPVSR |
| SEQ ID NO: 20 | IIKKYQLQFR |
| SEQ ID NO: 21 | AQIFQRDQSLVEDMNSMVR |
| SEQ ID NO: 22 | LIRYNNFLHDPLSLCEACIPKP |
| SEQ ID NO: 23 | SVLLDAASGQLR |
| SEQ ID NO: 24 | DQSLVEDMNSMVR |
| SEQ ID NO: 25 | DLLVAHNTWNSYQNMLR |
| SEQ ID NO: 26 | YNNFLHDPLSLCEACIPKPNA |
| SEQ ID NO: 27 | RMSMLAASGPTWDQLPPFQ |
| SEQ ID NO: 28 | SMLHMGQPDLWTFSPISVP |
| SEQ ID NO: 29 | MSMLAASGPTWDQLPPFQ |
| SEQ ID NO: 30 | VRSVLLDAASGQLR |
| SEQ ID NO: 31 | QNLDPPVSR |

As used herein, the phrase “percent fatty acid ester hydrolysis” means the molar proportion of fatty acid ester that has been hydrolyzed. Since hydrolysis of a fatty acid ester results in the release of a free fatty acid, the percent fatty acid ester hydrolysis can be determined by measuring the free fatty acid in a sample. Therefore, percent fatty acid ester hydrolysis may be determined by calculating moles of free fatty acid divided by the sum of moles of free fatty acid plus moles of fatty acid ester. In the case of percent hydrolysis of polysorbate 80 or polysorbate 20, that number may be determined by calculating the moles of free fatty acid, and dividing by the total moles of remaining polysorbate plus moles of free fatty acid.

The term “esterase inhibitor” means any chemical entity that reduces, inhibits, or blocks the activity of an esterase. The applicants envision that the inclusion of an esterase inhibitor in a protein formulation containing a fatty acid ester surfactant may help maintain protein stability and help reduce SVP formation. Common esterase inhibitors known in the art include orlistat (tetrahydrolipistatin; an inhibitor of carboxylesterase 2 and lipoprotein lipase), diethylumbelliferyl phosphate (a cholesterol esterase [lipsase A] inhibitor), URB602 ([1-1′-biphenyl]-3-tl-carbamicacid cyclohexyl ester; a monoacylglycerol lipase inhibitor), and 2-butoxyphenylboronic acid (an inhibitor of hormone-sensitive lipase). The inclusion of an esterase inhibitor during purification of a protein of interest or in the final formulation may prevent or slow the hydrolysis of non-ionic detergents like polysorbate 80, which in turn are expected to prevent or reduce subvisible particle formation. However, the inclusion of an esterase inhibitor may also negatively affect the functioning of the active ingredient, or other ingredients, in the final formulation.

The term “buffer” means a buffering solution or a buffering agent that stabilizes the pH of a solution. A buffer generally comprises a weak acid and its conjugate base, or a weak base and its conjugate acid. Buffering of a protein solution at or close to the optimal pH helps to ensure proper protein folding and function. The best buffer can be identified, for example, by measuring the thermodynamic stability (DSC), and high molecular weight variants (SEC) and charge variants (CEX) of the protein (e.g., antibody) solution at various pHs following accelerated storage/incubation. Measuring the circular dichroism of the protein (e.g., antibody) solution at various pHs may also assist in identifying a buffer. Circular dichrosim (CD) is one method used to determine structural changes (unfolding) of a protein (S. Beychok, “Circular dichroism of biological macromolecules,” Science 154(3754):1288-99 (1966); Kemmer and Keller, “Nonlinear least-squares data fitting in Excel spreadsheets,” Nat Protoc. 5(2):267-81 (2010)). Some proteins possess the ability to act as buffers (i.e., so called “self-buffering”) and therefore may not require the addition of an exogenous buffer to maintain stable pH (Gokarn et al., “Self-buffering antibody formulations,” J Pharm Sci. 97(8):3051-66 (2008)). Examples of commonly used buffers are listed in Table 2. For a more complete discussion of buffers in biological solutions, see Irwin H. Segel, Biochemical Calculations (2^{nd} ed. 1976), or Remington, The Science and Practice of Pharmacy 244 (Paul Beringer et al. eds., 21^{st} ed. 2006).

TABLE 2

| Buffering Agent | pKa | Useful pH |
|---|---|---|
| Histidine | 1.82, 6.0, 9.17 | 5.5-7.4 |
| Citrate | 3.13, 4.76, 6.40 | 2.1-7.4 |
| Glycine | 2.35, 9.78 | 2.2-3.6, 8.8-10.6 |
| Acetate | 4.8 | 3.8-5.8 |
| Phosphate | 7.2 | 6.2-8.2 |
| Succinate | 4.21, 5.64 | 3.2-6.5 |
| Tris | 8.06 | 7.5-9.0 |
| HEPES | 7.48 | 6.8-8.2 |
| MOPS | 7.20 | 6.5-7.9 |
| PIPES | 6.76 | 6.1-7.5 |

The term “thermal stabilizer” means an excipient or other additive included in a biopharmaceutical formulation to provide protection to the protein against thermal degradation, denaturation, and erosion of biological activity. Generally, a thermal stabilizer helps maintain the protein (e.g., antibody) in its native conformation and prevent aggregation under conditions of thermal stress. Thermal stress may occur from freeze-thaw cycling, exposure to high temperatures, or extensive storage time. Thermal stabilizers include sugars and other carbohydrates, sugar alcohols and polyols like polyethylene glycol, and amino acids like glycine. Examples of sugars or sugar alcohols useful as a thermal stabilizer include sucrose, trehalose and mannitol.

The term “hydrophobic interaction media” means a combination of a support structure and a hydrophobic moiety, wherein the hydrophobic moiety is affixed to the support structure. The media can be in the form of chromatography media, e.g., beads or other particles held in a packed bed column format, in the form of a membrane, or in any format that can accommodate a liquid comprising a protein of interest and contaminants. Thus, support structures include agarose beads (e.g., sepharose), silica beads, cellulosic membranes, cellulosic beads, hydrophilic polymer beads, and the like. The hydrophobic moiety binds to hydrophobic molecules and hydrophobic surfaces of proteins. The degree of hydrophobicity of the media can be controlled by selecting the hydrophobic moiety. Hydrophobic interaction media is employed in a process known as hydrophobic interaction chromatography (HIC) and is used to separate proteins of interest from product and process related contaminants. When the protein of interest is manufactured in and/or purified from host cells, the product and process related contaminants are referred to as host cell proteins (HCP). HCPs from Chinese hamster ovary (CHO) cells, a common biotherapeutic manufacturing host cell, can be referred to as CHOPs (Chinese hamster ovary proteins). In some cases, a mixture containing the protein of interest (POI) and HCPs are applied to the HIC media in a buffer designed to promote binding of hydrophobic groups in the POI to the hydrophobic moiety of the HIC medium. The POI sticks to the HIC media by binding the hydrophobic moiety, and some HCPs fail to bind and come out in the wash buffer. The POI is then eluted using a buffer that promotes dissociation of the POI from the HIC hydrophobic moiety, thereby separating the POI from unwanted HCPs.

In some cases, the HIC hydrophobic moiety binds some contaminants such as HCPs, and the POI is collected from the HIC flow-through.

In some cases affinity chromatography designed to bind specific proteins having lipophilic attributes is employed in lieu of or in concert with HIC. Since some esterases, such as lipases in general, or phospholipases in particular, bind to triglycerides or phospholipids, molecules that mimic those lipids may be used to capture esterases. For example, “myristoylated ADP ribosylating factor 1” (a.k.a. “myrARF1”) can be used to capture a lipase and allow the POI to remain unbound and flow through.

As used herein, the term “container” is meant to include a primary packaging component such as a syringe (as in a pre-filled syringe), a vial (for example a 2.5 mL glass vial for storing a biopharmaceutical formulation), or any vessel or means to contain a soild, liquid or gaseous substance. Here, the term “container” is used to refer inter alia to the vessel containing a biopharmaceutical formulation, as that term is used by the FDA and USP in its guidance on limitations for subvisible particles (United States Pharmacopeia and National Formulary (USP 40-NF 28), <787> Subvisible Particulate Matter in Therapeutic Protein Injections).

The terms “composition,” “formulation,” and “formulated drug substance” (FDS) as used in the present disclosure refer to a combination of two or more pharmaceutical ingredients for inclusion in a drug product. A composition, formulation, or FDS may be, for example, a liquid composition including an active pharmaceutical ingredient, such as an antibody, and an excipient, such as a stabilizer or surfactant. A composition, formulation, or FDS may include multiple excipients. A composition, formulation, or FDS may also include other constituents, such as proteins co-purified with an antibody.

The term “drug product” (DP) as used in the present disclosure refers to a dosage form comprising an amount of a FDS for packaging, shipment, or administration. For example, a drug product may be a pre-filled syringe holding a volume of FDS for administration to a patient.

As has been discussed above, it is hypothesized that HCPs such as PLBL2, which copurify with some POIs, exhibit esterase-like activity on fatty acid esters in polysorbates that are used in formulations and drug products with those POIs. This esterase-like behavior is thought to result in formation of free fatty acids that then may aggregate to form SVPs. While HIC and/or affinity chromatography may be used to purify a POI and remove HCPs from a drug product or formulation, thus reducing esterase-like behavior on fatty acid esters, the addition of a HIC or affinity chromatography step requires adding equipment (e.g., hydrophobic interaction media), materials, preparation, protocol, and protocol validation to a drug product's manufacturing process, meaning added time, resources, experimentation, and costs. Therefore, it is desirable to have an alternative method of decreasing SVP formation in formulations and drug products including a POI, a polysorbate, and a co-purified HCP.

It has been found that FDS and drug products which include a POI and a polysorbate 80 having a high percentage (e.g., >98%) of oleic acid ester content exhibit less measurable formation of SVPs over time than, e.g., FDS and drug products including polysorbate 80 having a relatively lower percentage (e.g., 70% or 86-87%) of oleic acid ester content. This is the case even when the POI is not subjected to HIC or affinity chromatography to remove HCPs that have esterase-like behavior on fatty acid esters.

Embodiments of the present disclosure relate to FDS and drug products including a POI (such as an antibody) and polysorbate 80 having >98% oleic acid ester content, where the POI has not been subjected to a HIC or affinity chromatography step to remove HCPs having esterase-like behavior. In some embodiments of the present disclosure, FDS and drug products exhibit formation of fewer than 3,000 particles having a diameter of 10 m or larger when stored in a container at a temperature of, e.g., 5° C. for at least 6 months. In some embodiments, FDS and drug products exhibit formation of fewer than 2,000, 1,500, 1,000, 800, 600, 500, 400, 300, 290, 275, 270, or 250 particles having a diameter of 10 m or larger when stored in a container at a temperature of, e.g., 5° C. for at least 6 months. In some aspects, embodiments of the present disclosure relate to methods of preparing such FDS and drug products.

In embodiments of the present disclosure, the FDS or drug product includes a POI. In some embodiments, the POI is an antibody, such as a human monoclonal antibody. In some embodiments, the POI is an immunoglobulin, such as IgG. In some embodiments, the protein is an IgG 1, an IgG 2, an IgG 3, or an IgG 4. In some embodiments, the FDS or drug product includes more than one POI (e.g., the FDS or drug product includes a co-formulation of two or more POIs).

In embodiments of the present disclosure, the POI may have been purified by a purification step known in the art. For example, if the POI is an immunoglobulin, it may have been purified using a Protein A or Protein G affinity purification step. In some embodiments, one or more HCPs or other impurities may have been copurified with the POI during this purification step. For example, in some embodiments, the FDS or drug product includes an esterase copurified with the POI. In some embodiments, the esterase is a phospholipase B-like protein, such as PLBL2.

In embodiments of the present disclosure, the concentration of the POI in the FDS or drug product may range from about 40 mg/mL to about 250 mg/mL, such as, for example, between about 50 mg/mL and about 160 mg/mL, between about 80 mg/mL and 100 mg/mL, between about 100 mg/mL and 160 mg/mL, between about 125 mg/mL and 155 mg/mL.

In embodiments, the FDS or drug product includes an amount of a surfactant or stabilizer. In some embodiments, the surfactant or stabilizer is a polysorbate 80 including a mix of fatty acid esters, and which has at least a 97%, 98%, or 99% content of oleic acid esters. In some embodiments, the surfactant or stabilizer is polysorbate 80 including a mix of fatty acid esters, and which has a >98% content of oleic acid esters. In further embodiments, the surfactant or stabilizer is a polysorbate 80 including a mix of fatty acid esters, and which has a ≥99% content of oleic acid esters. In embodiments, a concentration of the surfactant or stabilizer in the FDS or drug product is between 0.005% and 1.00% (w/v), such as 0.5% (w/v).

In some embodiments, a volume of the FDS or drug product is between about 0.25 mL and 3 mL, such as 0.25 mL, 0.5 mL, 1 mL, 1.5 mL, 2 mL, 2.25 mL, 2.5 mL, or 3 mL. In some embodiments, the drug product includes a volume of the FDS packaged in a container.

In some embodiments, the FDS or drug product includes additional excipients, such as a buffer, a thermal stabilizer, or an esterase inhibitor.

In some embodiments, the FDS or drug product is stored at a temperature of about 2-8° C. for at least 6 months. In other embodiments, the FDS or drug product is stored at a temperature of, e.g., about 5° C., 15° C., 22° C., 24° C., or 30-50° C., such as about 35° C., 40° C., 45° C., or 50° C.

In some embodiments, the FDS or drug product is stored for up to, e.g., 2-4 weeks, 2 months, 3 months, 4 months, 5 months, 6 months, 12 months, 18 months, 24 months, or 36 months. For example, in some embodiments, the FDS or drug product is stored at a temperature of about 5° C. for up to 24 months. In other embodiments, the FDS or drug product is stored at a temperature of about 30-50° C. for up to 5 months.

### EXAMPLES

### Example 1

The storage stability of an IgG4 antibody drug product prone to forming free fatty acid-based subvisible particulates due to degradation of polysorbate by a co-purified host cell protein (HCP) lipase was evaluated in different DP samples. Each DP sample had a volume of 2.136 mL, contained the same concentration of the IgG4 antibody (150 mg/mL), and 0.2% (w/v) of one of several lots of PS80. Each lot of PS80 lots had one of three different percentage contents of oleic acid ester (70%, 87%, and ≥99%). The table below summarizes the percentage content of oleic acid ester in the PS80 in each FDS sample.

TABLE 3

| | % content of oleic acid |
|---|---|
| Name | ester in PS80 (Lot) |
| DP A | 87% |
| DP B | ≥99% (Lot 1) |
| DP C | ≥99% (Lot 2) |
| DP D | 70% (Lot 1) |
| DP E | 70% (Lot 2) |
| DP F | 70% (Lot 3) |

The DP samples were stored at 2-8° C. in glass pre-filled syringes for up to 24 months. Particulates were measured in each DP sample every six months for a total of 24 months, by both the microscopic method and micro-flow imaging (MFI).

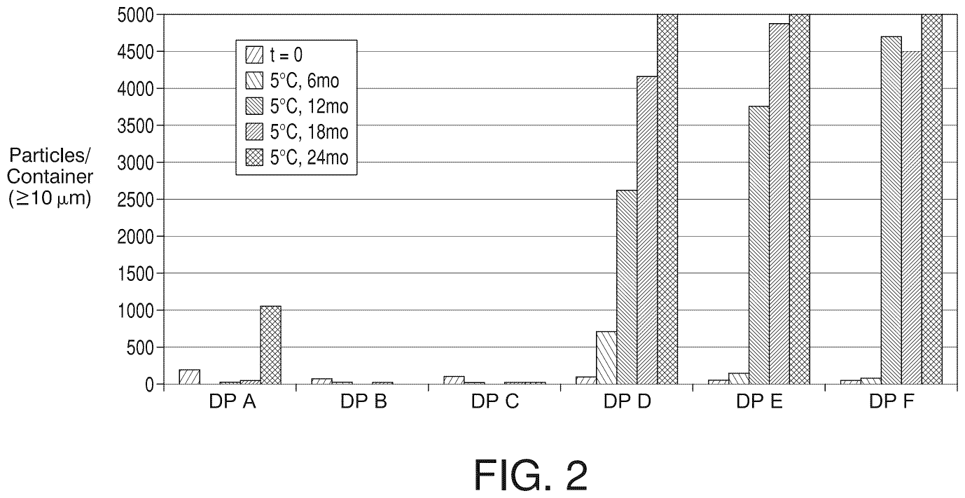

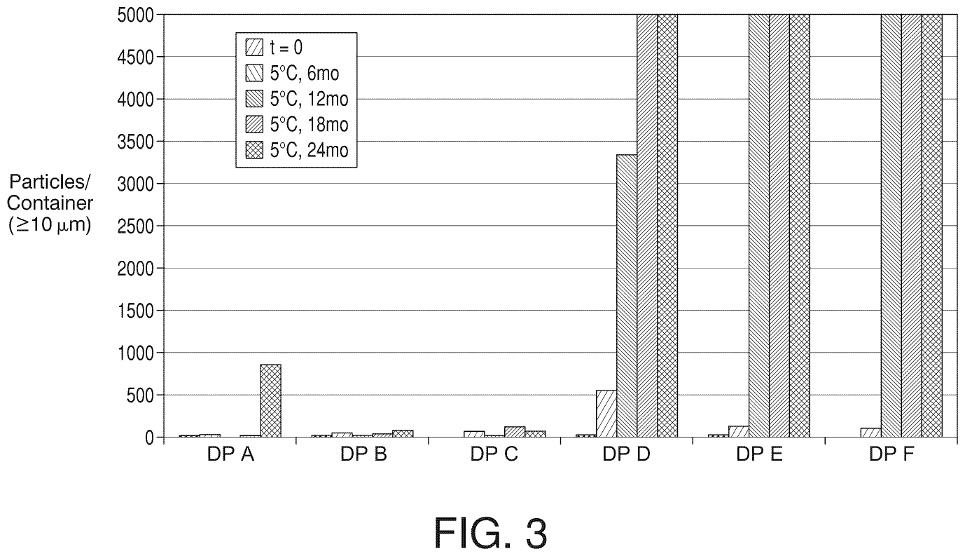

FIG. 2 shows, in chart form, the number of SVPs per container having a diameter of ≥10 m, as measured by the microscopic method. FIG. 3 shows, in chart form, the number of SVPs per container having a diameter of ≥10 m, as measured by MFI. As is shown in FIGS. 2 and 3, DP B and DP C (the two DP samples containing PS80 having a ≥99% content of oleic acid esters) displayed the lowest numbers of SVPs across the full 24-month period, as measured by both microscopy (FIG. 2) and MFI (FIG. 3). DP A, containing PS80 having an 87% content of oleic acid esters, displayed the next lowest number of subvisible particulates across the 24-month period (in particular, showing between 800 and 1200 particles by 24 months). DPs D, E, and F all showed well over 3000 particles per container (as measured by both methods) by at least the 18-month mark.

It was hypothesized that the lower numbers of particles in DPs A, B, and C (as compared to the more numerous particles in DPs D, E, and F) were a result of the use of PS80 having a higher percentage content of oleic acid (or long-chain fatty acid) esters. Oleic acid is a longer chain fatty acid, with one unsaturated bond (see FIG. 1). Therefore, it has a sub-ambient melting temperature of about 13° C. A precursor to subvisible and visible free fatty acid (FFA) particulate formation is the agglomeration of individual FFA chains into aggregates, which then precipitate in the form of particles. Oleic acid may be generated during storage of the formulations at 5° C. by, e.g., enzymatic hydrolysis of the fatty acid esters in polysorbate 80. This oleic acid may form SVPs, but due to its low melting temperature, such particles are more likely to exist as an oily liquid in protein formulations at room temperature (about 22° C.) where analysis is performed, and therefore does not persist as subvisible particulates at room temperature. As a contrast, higher amounts of non-oleic acid ester content in the formulation will lead to formation of their corresponding FFA upon hydrolysis, and due to their higher melting temperatures the subvisible and visible amorphous particulates thus formed persist at ambient temperature during analysis.

Additionally, oleic acid esters are better solubilizing/stabilizing agents than esters of shorter chain fatty acids due to their (oleic acid esters') higher hydrophobicity, which enables oleic acid esters to solubilize free fatty acid and protein particulates thereby maintaining product stability. Therefore, polysorbate 80 with higher contents of oleic acid esters (>98%) can provide improved stability to protein formulations and drug products as compared to polysorbate 80 with lower contents of oleic acid esters.

### Example 2

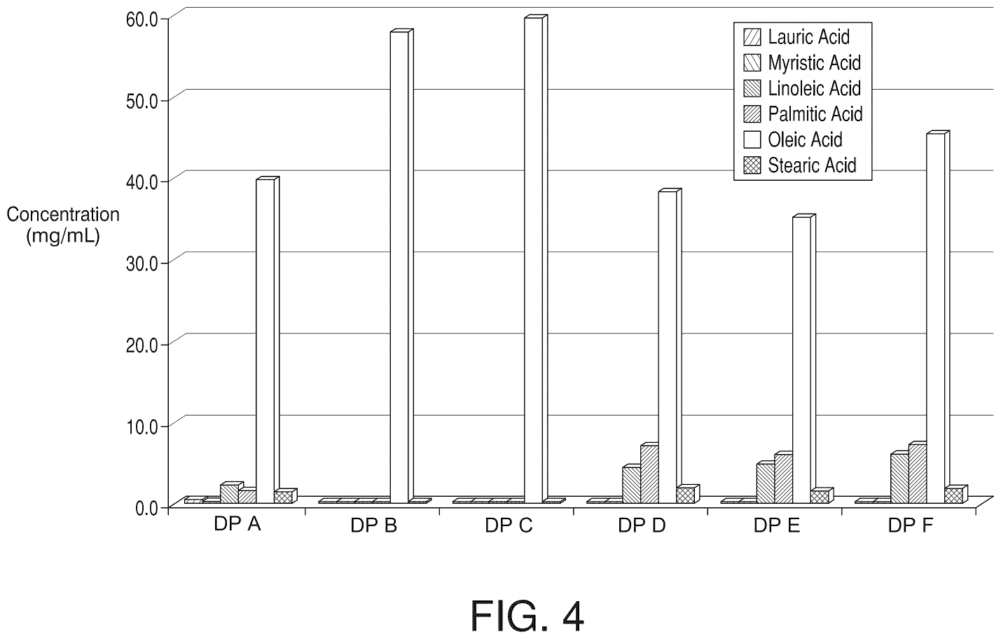

A concentration of each type of free fatty acid (in micrograms/mL) in each sample DP (DP A-F) was evaluated after storage of the samples at 5° C. for 18 months. Sample DP A-F were prepared as described in Example 1. Free fatty acid concentrations were measured at 18 months by LC-MS. FIG. 4 displays the results in chart form. As depicted, DPs B and C (the two DP samples containing PS80 having a ≥99% content of oleic acid esters) displayed the highest concentration of oleic acid, and the lowest concentrations of other FFAs. This indicates the homogeneity of the FFAs (i.e., oleic acids) in DPs B and C.

1. A formulation, comprising:
at least 100 mg/mL of an IgG antibody; and
a mixture of fatty acid esters of polyoxyethylene sorbitan, the polyoxyethylene sorbitan having a content of greater than 98% oleic acid esters,
wherein the mixture of fatty acid esters of polyoxyethylene sorbitan is present in an amount ranging from about 0.005% to 1.00%, relative to the total weight of the formulation.

2. The formulation of claim 1, comprising at least 150 mg/mL of the IgG antibody.

3. The formulation of claim 1, wherein the mixture of fatty acid esters of polyethylene sorbitan is polysorbate 80.

4. The formulation of claim 1, wherein the IgG antibody is an IgG4 antibody.

5. The formulation of claim 1, further comprising an esterase.

6. The formulation of claim 5, wherein the esterase is a phospholipase B-like 2 protein.

7. The formulation of claim 1, wherein the polyoxyethylene sorbitan has a content of at least 99% oleic acid esters.

8. The formulation of claim 1, wherein the mixture of fatty acid esters of polyethylene sorbitan is present in an amount ranging from about 0.5% to 1.00%, relative to the total weight of the formulation.

9. A drug product including:
at least 100 mg/mL of an IgG antibody; and
a mixture of fatty acid esters of polyoxyethylene sorbitan, the polyoxyethylene sorbitan having a content of greater than 98% oleic acid esters,
wherein the mixture of fatty acid esters of polyethylene sorbitan is present in an amount ranging from about 0.005% to 1.00%, relative to the total weight of the formulation.

10. The drug product of claim 9, wherein fewer than 3000 particles having a diameter of 10 microns or greater are detectable in the drug product by flow imaging microscopy or membrane microscopy, after the drug product has been stored at a temperature of between 30° C. and 50° C. for between 1 and 5 months.

11. The drug product of claim 9, wherein fewer than 3000 particles having a diameter of 10 microns or greater are detectable in the drug product by one of flow imaging microscopy or membrane microscopy, after the drug product has been stored at a temperature of between 2° C. and 8° C. for between 18 and 36 months.

12. The drug product of claim 9, comprising at least 150 mg/mL of the IgG antibody.

13. The drug product of claim 9, wherein the IgG antibody is an IgG4 antibody.

14. The drug product of claim 9, wherein the mixture of fatty acid esters of polyethylene sorbitan is polysorbate 80.

15. The drug product of claim 9, wherein the IgG antibody is an IgG4 antibody.

16. The drug product of claim 9, wherein a volume of the drug product is between about 0.25 mL and about 3 mL.

17. The drug product of claim 9, wherein polyoxyethylene sorbitan has a content of at least 99% oleic acid esters.

18. The drug product of claim 9, further comprising a phospholipase B-like 2 protein.

19. The drug product of claim 9, further comprising an esterase.

20. The drug product of claim 9, wherein the mixture of fatty acid esters of polyethylene sorbitan is present in an amount ranging from about 0.5% to 1.00%, relative to the total weight of the drug product.


SEQUENCE LISTING (31 sequences)

SEQ ID NO: 1 - protein, 17 aa, Cricetulus griseus
DLLVAHNTWNSYQNMLR

SEQ ID NO: 2 - protein, 22 aa, Cricetulus griseus
LIRYNNFLHDPLSLCEACIPKP

SEQ ID NO: 3 - protein, 12 aa, Cricetulus griseus
SVLLDAASGQLR

SEQ ID NO: 4 - protein, 13 aa, Cricetulus griseus
DQSLVEDMNSMVR

SEQ ID NO: 5 - protein, 17 aa, Cricetulus griseus
QFNSGTYNNQWMIVDYK

SEQ ID NO: 6 - protein, 20 aa, Cricetulus griseus
QGPQEAYPLIAGNNLVFSSY

SEQ ID NO: 7 - protein, 19 aa, Cricetulus griseus
SMLHMGQPDLWTFSPISVP

SEQ ID NO: 8 - protein, 21 aa, Cricetulus griseus
YNNFLHDPLSLCEACIPKPNA

SEQ ID NO: 9 - protein, 13 aa, Cricetulus griseus
LALDGATWADIFK

SEQ ID NO: 10 - protein, 13 aa, Cricetulus griseus
LSLGSGSCSAIIK

SEQ ID NO: 11 - protein, 13 aa, Cricetulus griseus
YVQPQGCVLEWIR

SEQ ID NO: 12 - protein, 19 aa, Cricetulus griseus
RMSMLAASGPTWDQLPPFQ

SEQ ID NO: 13 - protein, 12 aa, Cricetulus griseus
SFLEINLEWMQR

SEQ ID NO: 14 - protein, 22 aa, Cricetulus griseus
VLTILEQIPGMVVVADADKTED

SEQ ID NO: 15 - protein, 14 aa, Cricetulus griseus
VRSVLLDAASGQLR

SEQ ID NO: 16 - protein, 16 aa, Cricetulus griseus
LTLLQLKGLEDSYEGR

SEQ ID NO: 17 - protein, 18 aa, Cricetulus griseus
MSMLAASGPTWDQLPPFQ

SEQ ID NO: 18 - protein, 9 aa, Cricetulus griseus
VTSFSLAKR

SEQ ID NO: 19 - protein, 9 aa, Cricetulus griseus
QNLDPPVSR

SEQ ID NO: 20 - protein, 10 aa, Cricetulus griseus
IIKKYQLQFR

SEQ ID NO: 21 - protein, 19 aa, Cricetulus griseus
AQIFQRDQSLVEDMNSMVR

SEQ ID NO: 22 - protein, 22 aa, Cricetulus griseus
LIRYNNFLHDPLSLCEACIPKP

SEQ ID NO: 23 - protein, 12 aa, Cricetulus griseus
SVLLDAASGQLR

SEQ ID NO: 24 - protein, 13 aa, Cricetulus griseus
DQSLVEDMNSMVR

SEQ ID NO: 25 - protein, 17 aa, Cricetulus griseus
DLLVAHNTWNSYQNMLR

SEQ ID NO: 26 - protein, 21 aa, Cricetulus griseus
YNNFLHDPLSLCEACIPKPNA

SEQ ID NO: 27 - protein, 19 aa, Cricetulus griseus
RMSMLAASGPTWDQLPPFQ

SEQ ID NO: 28 - protein, 19 aa, Cricetulus griseus
SMLHMGQPDLWTFSPISVP

SEQ ID NO: 29 - protein, 18 aa, Cricetulus griseus
MSMLAASGPTWDQLPPFQ

SEQ ID NO: 30 - protein, 14 aa, Cricetulus griseus
VRSVLLDAASGQLR

SEQ ID NO: 31 - protein, 9 aa, Cricetulus griseus
QNLDPPVSR